(19) United States
(12) Patent Application Publication
(10) Pub. No.: US 2021/0128885 A1
(43) Pub. Date: May 6, 2021

(54) MEDICAL DEVICES WITH REINFORCED WIRES

(71) Applicants: STRYKER CORPORATION, Kalamazoo, MI (US); STRYKER EUROPEAN OPERATIONS LIMITED, Carrigtwohill (IE)

(72) Inventor: Jeffrey LU, Kalamazoo, MI (US)

(21) Appl. No.: 16/676,338

(22) Filed: Nov. 6, 2019

(51) Int. Cl. A61M 25/09

(52) U.S. Cl. CPC A61M 25/09; A61M 2025/09083

(57) ABSTRACT

A medical device includes: a wire having a proximal end, a distal end, and a body extending between the proximal end and the distal end; and a coil disposed around a segment of the wire, wherein the coil comprises a proximal coil end, a distal coil end, and a coil body extending between the proximal coil end and the distal coil end; wherein the coil body comprises loops made from an elongated member having a cross-section, wherein the cross-section of the elongated member comprises a first side facing the wire, and a second side being opposite the first side and facing away from the wire; and wherein the second side of the cross-section of the elongated member has a higher curvature than the first side of the cross-section of the elongated member.

### FIELD

The present disclosure relates generally to minimally invasive medical devices, and more specifically to minimally invasive medical devices with reinforced wires, such as guidewires and pushwires for delivering objects.

### BACKGROUND

The use of intravascular implants, such as stents, stent grafts, flow-diverters, aneurysm occlusive devices, vena cava filters, etc., has become an effective method for treating many types of vascular disease. In general, a suitable intravascular implantable device is inserted into the vascular system of the patient and navigated through the vasculature to a targeted implantation site using a delivery system.

Minimally invasive delivery systems include catheters, push or delivery wires, and the like, are percutaneously introduced into the patient's vasculature over a guidewire. Commonly used vascular application to access a target site in a patient involves inserting a guidewire through an incision in the femoral artery near the groin, and advancing the guidewire until it reaches the target site. Then, a catheter is advanced over the guidewire until an open distal end of the catheter is disposed at the target site. Simultaneously or after placement of the distal end of the catheter at the target site, an intravascular implant is advanced through the catheter via a delivery wire.

In certain applications, such as neurovascular, the guidewires and delivery wires are required to navigate tortuous and intricate vasculature. Thus, these wires (i.e., guidewires and delivery wires) should have suitable flexibility, kink resistance, pushability and torqueability to successfully navigate the vasculatures, such as cerebral and peripheral vasculature. Suitable flexibility and kink resistance of these wires allow them to navigate through a relatively tight bend without breaking or permanently deforming. Further, the forces applied at the proximal end of these wires should be transferred to the distal ends for suitable pushability (axial rigidity) and torqueability (rotation). Achieving a balance between these features is highly desirable. For example, the guidewires and/or delivery wires may comprise variable stiffness sections (e.g., varying ratio of material, including selective reinforcement, such as braids, coils, or the like) suitable to provide sufficient flexibility, kink resistance, pushability, and torqueability to allow navigation through vasculature.

In some cases, a guidewire or pushwire may include a coil disposed around a wire. The coil may fail and/or may behave unexpectedly during use, especially when the guidewire or pushwire is advanced through narrow bends and tortuous vasculature, thereby negatively impacting the overall performance of the delivery system.

### SUMMARY

A medical device includes: a wire having a proximal end, a distal end, and a body extending between the proximal end and the distal end; and a coil disposed around a segment of the wire, wherein the coil comprises a proximal coil end, a distal coil end, and a coil body extending between the proximal coil end and the distal coil end; wherein the coil body comprises loops made from an elongated member having a cross-section, wherein the cross-section of the elongated member comprises a first side facing the wire, and a second side being opposite the first side and facing away from the wire; and wherein the second side of the cross-section of the elongated member has a higher curvature than the first side of the cross-section of the elongated member.

Optionally, at least a part of the first side comprises a rectilinear profile.

Optionally, at least a part of the second side comprises a curvilinear profile.

Optionally, the first side has a width, and the cross-section of the elongated member has a height that is perpendicular to the width, and wherein the width is at least two times the height.

Optionally, the height is a dimension of a lateral side of the cross-section, the lateral side having a rectilinear profile and is configured to abut against one of the loops of the coil.

Optionally, the cross-section of the elongated member comprises a D-shape cross-section.

Optionally, the coil body is configured to form a smooth profile along a length of the wire as the wire undergoes bending so that no part of the coil body forms any sharp bend, and/or adjacent ones of the loops of the coil are configured to stay aligned as the wire with the coil undergoes bending.

Optionally, the medical device further includes a delivery catheter configured to house the wire with the coil.

Optionally, the distal coil end of the coil is proximal to the distal end of the wire.

Optionally, the wire with the coil forms a guidewire.

Optionally, the wire with the coil forms a pushwire configured to push an object.

Optionally, the object comprises a stent or an embolic coil.

A medical device includes: a wire having a proximal end, a distal end, and a body extending between the proximal end and the distal end; and a coil disposed around a segment of the wire, wherein the coil comprises a proximal coil end, a distal coil end, and a coil body extending between the proximal coil end and the distal coil end; wherein the coil body is configured to form a smooth profile along a length of the wire as the wire undergoes bending so that no part of the coil body forms any sharp bend, and/or wherein adjacent ones of the loops of the coil are configured to stay aligned as the wire with the coil undergoes bending.

Optionally, the coil body comprises loops made from an elongated member having a cross-section, wherein the cross-section of the elongated member comprises a first side facing the wire, and a second side being opposite the first side and facing away from the wire; and wherein the second side of the cross-section of the elongated member has a higher curvature than the first side of the cross-section of the elongated member.

Optionally, at least a part of the first side comprises a rectilinear profile.

Optionally, at least a part of the second side comprises a curvilinear profile.

Optionally, the first side has a width, and the cross-section of the elongated member has a height that is perpendicular to the width, and wherein the width is greater than the height, for example and without limitation, wherein the width is at least twice the height.

Optionally, the height is a dimension of a lateral side of the cross-section, the lateral side having a rectilinear profile and is configured to about against one of the loops of the coil.

Optionally, the cross-section of the elongated member comprises a D-shape cross-section.

Optionally, the medical device further includes a delivery catheter configured to house the wire with the coil.

Optionally, the wire with the coil forms a pushwire configured to push an object, and wherein the object comprises a stent or an embolic coil.

A medical device includes: a wire having a proximal end, a distal end, and a body extending between the proximal end and the distal end; and a coil disposed around a segment of the wire, wherein the coil comprises a proximal coil end, a distal coil end, and a coil body extending between the proximal coil end and the distal coil end; wherein the coil body comprises loops made from an elongated member having a cross-section, wherein the cross-section of the elongated member comprises a first side facing the wire, and a second side being opposite the first side and facing away from the wire; wherein the first side has a width, and the cross-section of the elongated member has a height that is perpendicular to the width, and wherein the width is at least two times the height; and wherein the height is a dimension of a lateral side of the cross-section, the lateral side having a rectilinear profile and is configured to abut against one of the loops of the coil.

Other aspects and features of embodiments will become apparent from the detailed description in view of the accompanying figures.

### BRIEF DESCRIPTION OF THE FIGURES

FIG. 1 illustrates a medical device comprising a wire with a coil;

FIG. 1A illustrates a detailed view of the medical device of FIG. 1;

FIG. 1B illustrates a cross-sectional side view of the medical device of FIG. 1, particularly showing the medical device not being bent;

FIG. 1C illustrates a cross-sectional side view of the medical device of FIG. 1, particularly showing the medical device being in a bending configuration;

FIG. 2 illustrates a constructed medical device with a coil made from an elongated member having a circular cross-section;

FIGS. 3A-3D illustrate the medical device of FIG. 2, particularly showing bending of the coil as the wire with the coil is being advanced inside a delivery catheter;

FIG. 4 illustrates the medical device of FIG. 2, particularly showing a condition of the coil after the wire with the coil has been advanced through the delivery catheter;

FIGS. 5 and 5A illustrate another constructed medical device having a coil made from elongated member having a rectangular cross-section;

FIG. 6 illustrates the medical device of FIG. 5, particularly showing the wire with the coil being advanced in a delivery catheter;

FIGS. 7 and 7A illustrate condition of the coil of the medical device of FIG. 5 after the wire with the coil has been advanced through the delivery catheter;

FIG. 8 illustrates a medical device having a wire and a coil;

FIG. 8A is a cross-sectional view of the medical device of FIG. 8;

FIG. 9 illustrates a perspective view of an elongated member of the coil of FIG. 8;

FIG. 9A is a detailed cross-sectional view of the elongated member of the coil of FIG. 8;

FIG. 10 illustrates a constructed medical device with a coil made from an elongated member having a D-shape cross-section;

FIGS. 11A-11F illustrate the medical device of FIG. 10, particularly showing bending of the coil as the wire with the coil is being advanced inside a delivery catheter;

FIG. 11G is a cross-sectional view of a coil made from an elongated member having a D-shape cross-section, and

FIG. 12 illustrates the medical device of FIG. 10, particularly showing the condition of the coil after being advanced within the delivery catheter.

### DETAILED DESCRIPTION

Various embodiments are described hereinafter with reference to the figures. The figures are not necessarily drawn to scale, and elements of similar structures or functions are represented by like reference numerals throughout the figures. It should also be understood that the figures are only intended to facilitate the description of the embodiments, and are not intended as an exhaustive description of the claimed inventions, or as a limitation on the scope thereof, which is defined only by the appended claims and their equivalents.

In addition, the respective illustrated embodiments need not have all of the depicted features. Also, an aspect or an advantage described in conjunction with a particular embodiment is not necessarily limited to that embodiment and can be practiced in any other embodiments even if not so illustrated, or if not so explicitly described.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

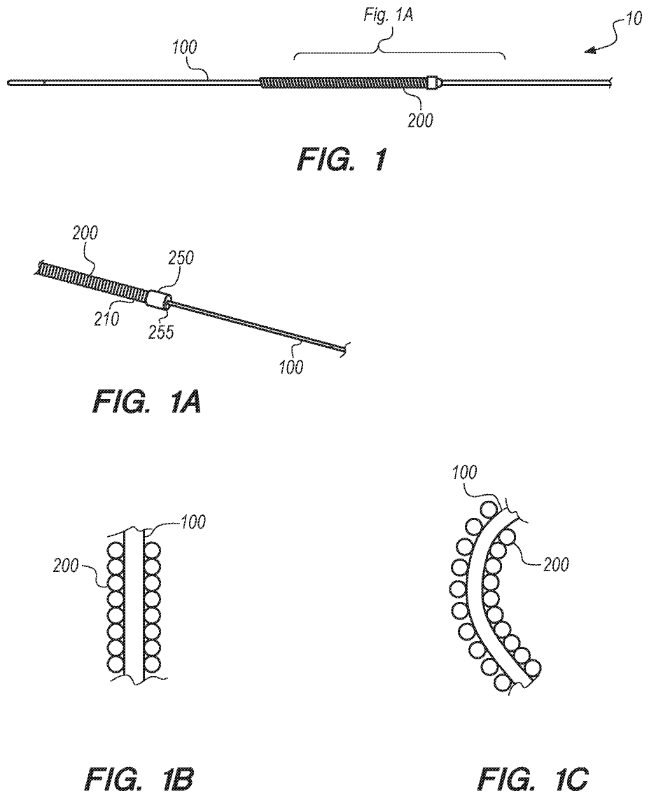

FIGS. 1 and 1A depict a medical device 10 having a wire 100 and a coil 200. The wire 100 is configured to be advanced through a delivery catheter lumen for delivering an implant (not shown) coupled to a distal end of the wire 100 to a targeted site in a patient's vasculature. As shown in the figures, the coil 200 is disposed around the wire 100, and may function to provide structural column strength. A radiopaque marker 250 is disposed on a distal portion 210 of the coil 200. An epoxy bond 255 may be used to attach the marker 250 to the wire 100. The marker 250 is in a form of a band, but in other embodiments, the marker 250 may have other shapes. In some cases, the wire 100 may include a re-sheading pad, a bumper, a coupler, a connector, or any of other components (not shown), for carrying an implant.

Referring to FIG. 1B, the coil 200 in the medical device 10 of FIG. 1 is composed of a round cross-sectional elongated member wound into a serious of loops that are disposed around the wire 100. Depending on the application, the coil 200 may be wound in a tight pitch. As shown in FIG. 1C, the coil 200 with the round cross-section is designed to assume a smooth curvilinear profile along a length of the wire 100 due to bending of the wire 100 as it is advanced through vasculatures in a patient. However, in some cases, a medical device constructed in accordance with the features (i.e., the circular cross section of the elongated member forming the coil 200) of FIG. 1B may unexpectedly perform in a manner that is different from that shown in FIG. 1C.

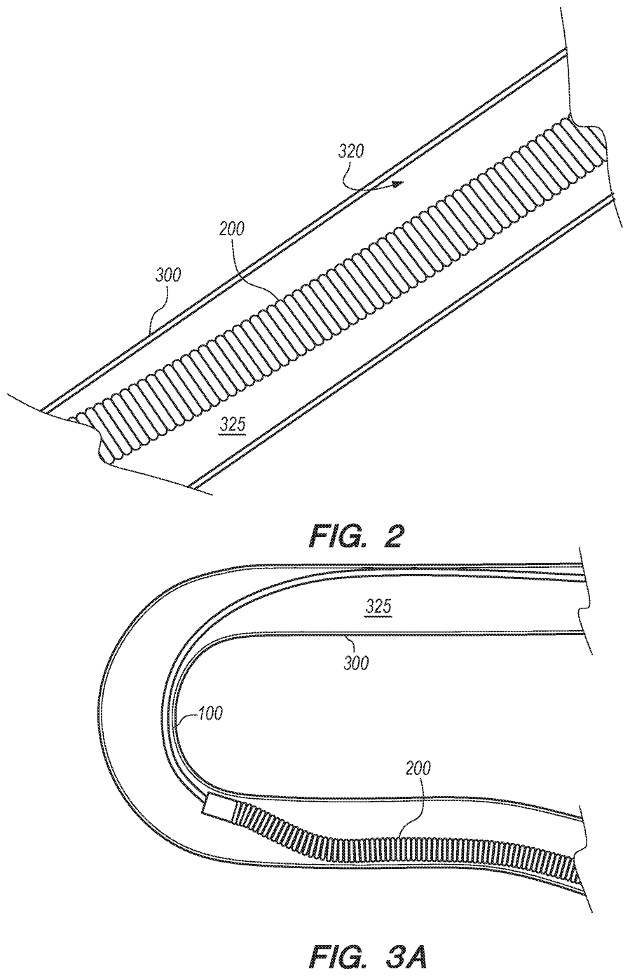

FIG. 2 illustrates a constructed medical device 10 with a coil 200 made from an elongated member having a circular cross-section. The medical device 10 of FIG. 2 is constructed in accordance with the features of FIG. 1B. As shown in FIG. 2, the coil 200 may be disposed within a delivery catheter 300 having an inner surface 320 defining a lumen 325.

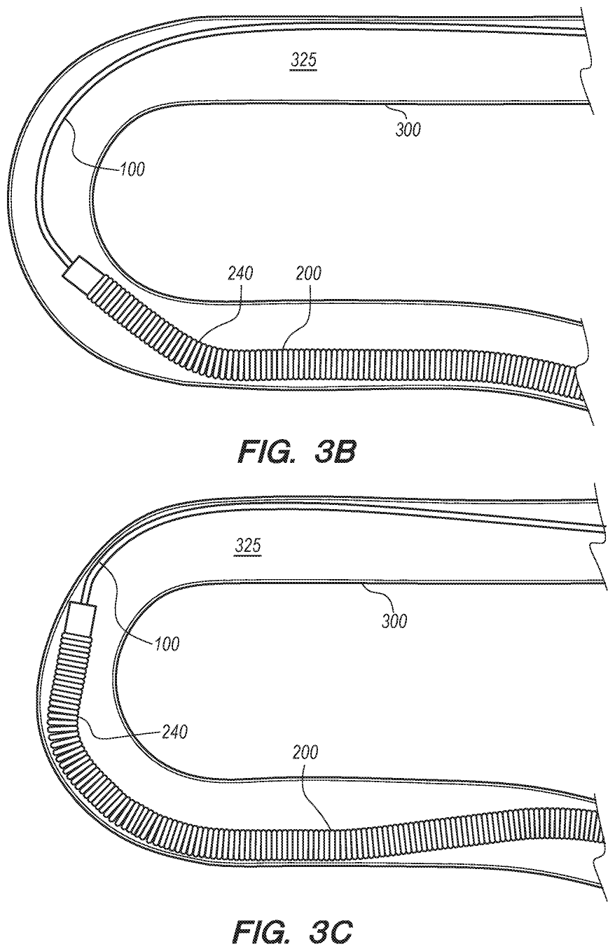

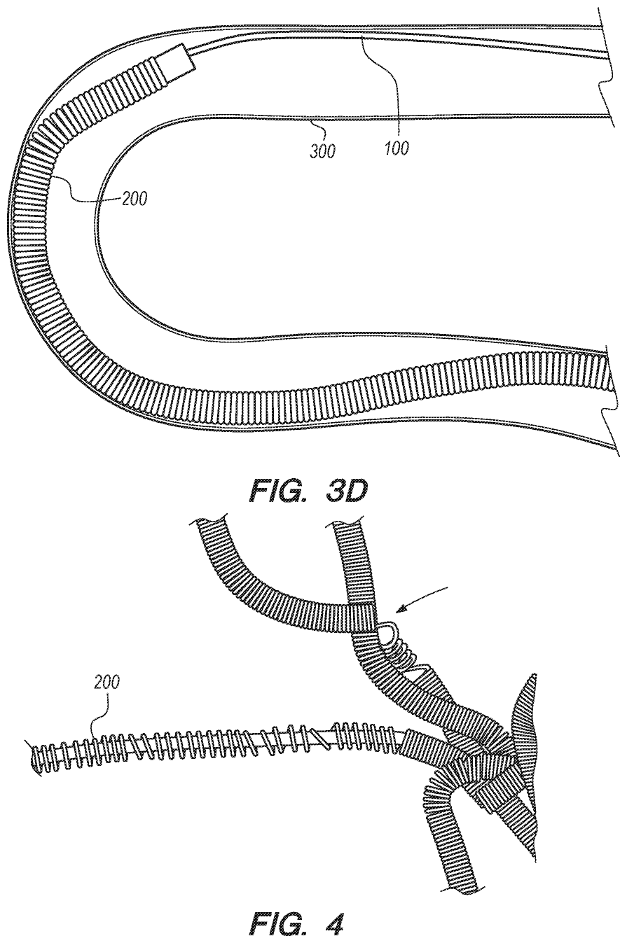

FIGS. 3A-3D illustrate a method of using the medical device 10 of FIG. 2, particularly showing bending of the coil 200, as the wire 100 with the coil 200 is being advanced inside a delivery catheter 300. In particular, FIG. 3A shows the wire 100 with the coil 200 being advanced through the lumen 325 of the delivery catheter 300, where the coil 200 is at a relatively straight section of the delivery catheter 300.

As shown in FIGS. 3B-3C, as the wire 100 with the coil 200 is advanced further, the coil 200 traverses through a more curvilinear section of the catheter 300 (i.e., through a bent with a higher curvature). This results in a concentrated stress imposed at the coil 200 at location 240, causing the coil 200 to unexpectedly form a kink or a plastic deformation. The damage (permanent deformation) to the coil 200 may be seen in FIG. 3D, as the coil 200 is exiting the bend into a more straight section of the delivery catheter 300. In some cases, the coil 200 may also twist and break, like that shown by the arrow in FIG. 4. Also, in some cases, during to the circular cross-section of the elongated member forming the coil, loops of the coil may slide out of alignment in a direction that is perpendicular to a longitudinal axis of the coil, causing some of the loops to rest over its two adjacent loops.

Thus, as shown above, despite the intended configuration shown in FIG. 1C, the actual device 10 constructed using the coil 200 with a circular cross-section according to the design of FIG. 1B may behave unexpectedly during use (i.e., in a manner that is different from the design shown in FIG. 1C), and may break unexpectedly.

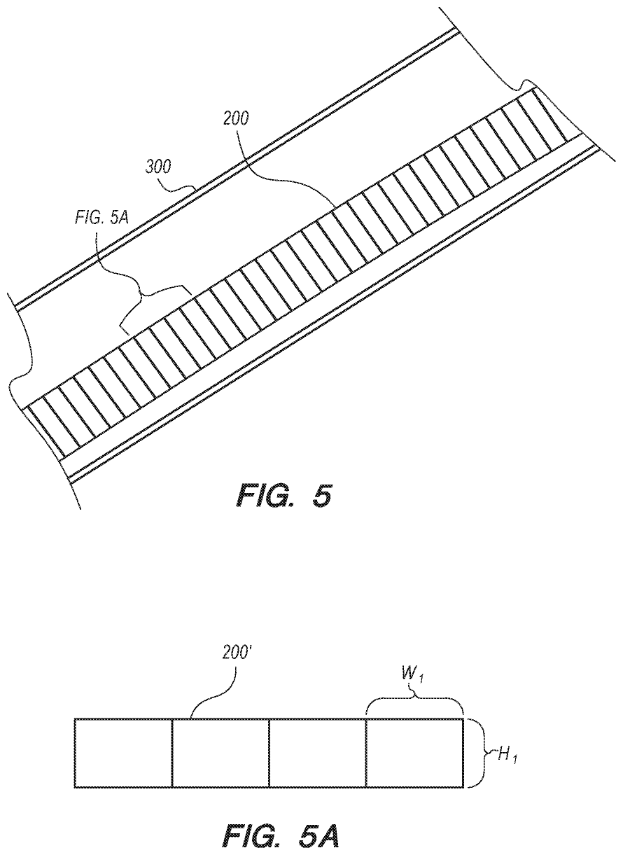

In other embodiments, the coil 200 may be formed with an elongated member having a rectangular cross-section. FIGS. 5 and 5A illustrate another constructed medical device 10 having a coil 200 made from an elongated member with a rectangular cross-section. The elongated member has a ribbon-like configuration in the illustrated embodiments. As shown in FIG. 5A, the rectangular cross-section of the elongated member has a width W measured along a longitudinal axis of the coil to be formed, and a height H measured in a direction that is perpendicular to the width W. In the illustrated example, the width W is 0.004″ (0.102 mm), and the height H is 0.002″ (0.051 mm).

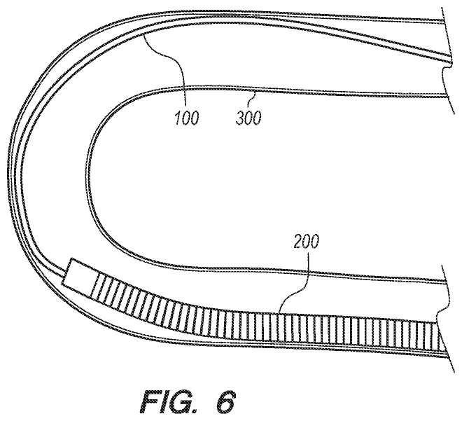

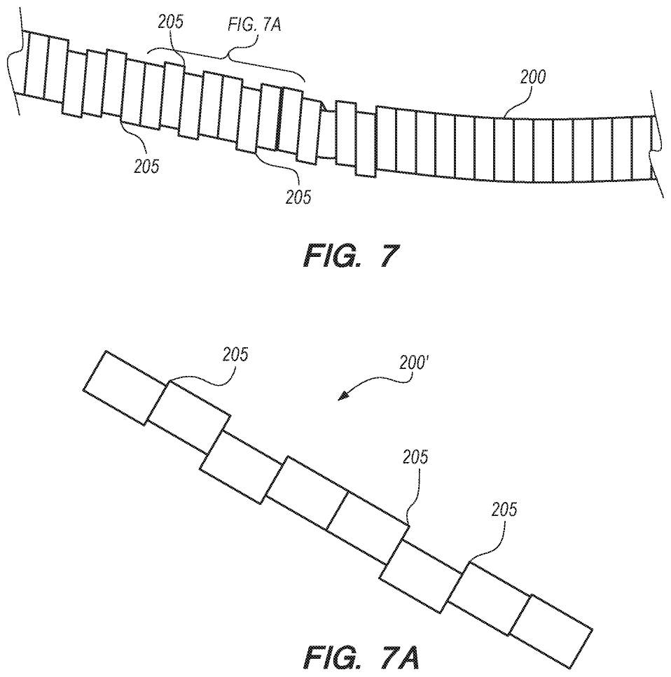

FIG. 6 illustrates the medical device 10 of FIG. 5, particularly showing the wire 100 with the coil 200 being advanced inside a delivery catheter 300. During use, axial force is applied at the proximal end to push the wire 100 with the coil 200 distally. This will cause the section of the wire 100 with the coil 200 to pass the bend shown in FIG. 6. As the coil 200 passes through the bend, compression is imposed on the loops at the compression side of the coil 200. FIGS. 7 and 7A illustrate condition of the coil 200 of the medical device 10 of FIG. 5 after the wire 100 with the coil 200 has been advanced through the bend. As discussed, bending of the coil 200 compresses the loops of the coil 200. Since the elongated member forming the coil 200 has a rectangular cross-section, compression of the coil 200 places concentrated stresses at the side edges 205 while the coil 200 is bending. In order to reduce such stresses, loops of the coil 200 move out of alignment (i.e., in a direction that is perpendicular to the longitudinal axis of the coil 200), creating an uneven outer surface of the coil 200. Due to such uneven outer surface of the coil 200, the exposed edges of the loops of the coil 200 act like serrated blades against the inner surface 325 of the delivery catheter 300, causing damaging (e.g., abrasion) to the catheter 300 and increasing the risk of undesirable failure of the delivery system. In some cases, the exposed sharp edges 205 may aggressively snag the inner surface 325 of the catheter 300. Also, since this “serrated” condition occurs while the coil 200 is bending in a tortuous vasculature, the exposed sharp edges 205 may cause trauma, rupture, piercing or significant damage to the blood vessel wall. It should be noted that the above “serrated” condition may also occur for other cross sections (e.g., square cross section, or other cross sections with right angles at the outer side of the coil) of the elongated member forming the coil 200.

As shown in the above examples, wire 100 with coil 200 having circular cross section, rectangular cross section, square cross section, or other cross sections with right angles at the outside of the coil 200, may behave in a manner that is different from an intended design, and/or may break during use. As a result, the wire 100 may fail to have sufficient column strength support or kink resistance necessary to deliver, for example, a low-profile implant. It would be desirable to provide guidewires and/or delivery wires with reinforcement coil that facilitates advancement through tortuous vasculature, while avoiding or minimizing breaking, abrasion, or other failure of the delivery system.

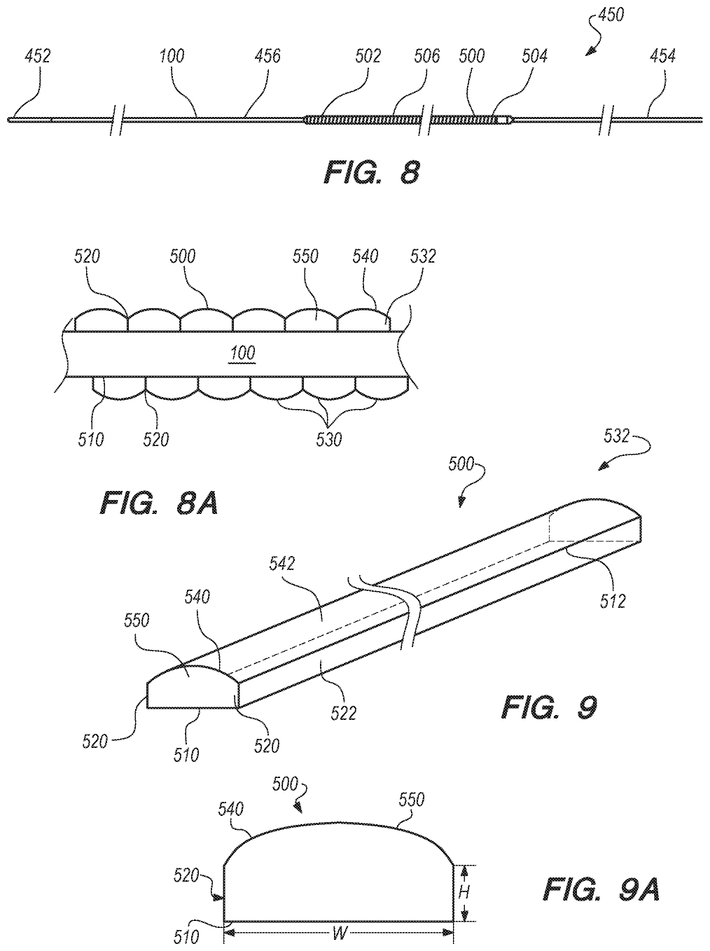

FIG. 8 illustrates a medical device 450 in accordance with some embodiments. FIG. 8A is a cross-sectional view of the medical device 450 of FIG. 8. As shown in FIGS. 8 and 8A, the medical device 450 includes a wire 100 having a proximal end 452, a distal end 454, and a body 456 extending between the proximal end 452 and the distal end 454. The wire 100 may be any shaft or elongated structure may from any materials, such as metal, allow, polymer, etc. The medical device 450 also includes a coil 500 disposed around a segment of the wire 100. As shown in the figure, the coil 500 has a proximal coil end 502, a distal coil end 504, and a coil body 506 extending between the proximal coil end 502 and the distal coil end 504. In the illustrated embodiments, the coil body 506 comprises loops 530 made from an elongated member 532 having a cross-section 550. As shown in FIGS. 8A, 9, and 9A, the cross-section 550 of the elongated member 532 forming the coil 500 has a first side 510 facing the wire 100, and a second side 540 being opposite the first side 510 and facing away from the wire 100. The second side 540 of the cross-section 550 of the elongated member 532 has a higher curvature than the first side 510 of the cross-section 550 of the elongated member 532.

As shown in FIGS. 9-9A, the first side 510 of the cross-section 550 is part of an inner-facing surface 512 configured to face the wire 100, the second side 540 of the cross-section 550 is part of an outward-facing surface 542, and the lateral side 520 of the cross-section 550 is a part of a lateral surface 522 configured to be disposed in contact with, or facing, adjacent loop 530. In some embodiments, the inner-facing surface 512 may be in contact with an exterior surface of the wire 100. In other embodiments, the inner-facing surface 512 may be facing, but spaced away from, the exterior surface of the wire 100.

In the illustrated embodiments, an entirety of the first side 510 comprises a rectilinear profile. In other embodiments, one or more parts of the first side 510 may have a curvilinear profile. For example, in other embodiments, opposite ends of the first side 510 may form a smooth curvilinear transition with respective lateral sides 520 of the cross-section 550. In addition, as shown in FIG. 9A, the first side 510 and an adjacent lateral side 520 forms a right angle. In other embodiments, the first side 510 and the lateral side 520 may form an acute angle.

Also, in the illustrated embodiments, at least a part of the second side 540 of the cross-section 550 comprises a curvilinear profile. In other embodiments, one or more parts of the second side 540 may have a rectilinear profile. In further embodiments, the second side 540 may have other profiles as long as an end of the second side 540 together with the adjacent lateral side 520 of the cross section 550 forms an angle that is not a 90° angle (e.g., any angle that is larger than 90°), or forms a smooth or curvilinear transition.

In the illustrated embodiments, the coil body 506 is configured to form a smooth profile along a length of the wire 100 as the wire 100 undergoes bending so that no part of the coil body forms any sharp bend (e.g., kink, plastic deformation, etc.). Alternatively or additionally, adjacent ones of the loops 530 of the coil 500 may be configured to stay aligned as the wire 100 with the coil 500 undergoes bending.

In some embodiments, the medical device 450 may further include a delivery catheter configured to house the wire 100 with the coil 500.

In the illustrated embodiments, the distal coil end 504 of the coil 500 is proximal to the distal end 454 of the wire 100. In other embodiments, the distal end 504 of the coil 500 may be distal to the distal end 454 of the wire 100.

In some embodiments, the wire 100 with the coil 500 forms a guidewire. In other embodiments, the wire 100 with the coil 500 forms a pushwire configured to push an object, which may be any implant, such as a stent or an embolic coil.

As shown in FIG. 9A, the cross-section 550 has a D-shape. In other embodiments, the cross-section may have other D-shapes that are different from the example shown. Also, as shown in the figure, the cross-section 550 of the elongated member 532 forming the coil 500 has a width W and a height H (measured in a direction that is perpendicular to the width W). In the illustrated embodiments, the height H is the height of the lateral side 520. In other embodiments, the height H may be the height of any part of the cross-section 550 that is in contact with an adjacent loop 530. In further embodiments, the height H may be the maximum height of the cross-section 550 measured in the direction that is perpendicular to the first side 510. Thus, the term “height” may refer to a dimension of a partial thickness of the cross-section 550, or a dimension of a complete thickness of the cross-section 550. In some embodiments, the width W is at least two times (e.g., 2×, 2.5×, 3×, 3.5×, 4×, etc.) the height H. In one specific example, the width W may be 0.004″ (0.102 mm) and the height H may be 0.002″ (0.051 mm). In another specific example, the width W of the cross-section 550 may be 0.002″ (0.051 mm) and the height H may be 0.001″ (0.025 mm). In other embodiments, the width W may be more than 0.004″ or less than 0.002″. Also, in other embodiments, the height H may be more than 0.002″ or less than 0.001″.

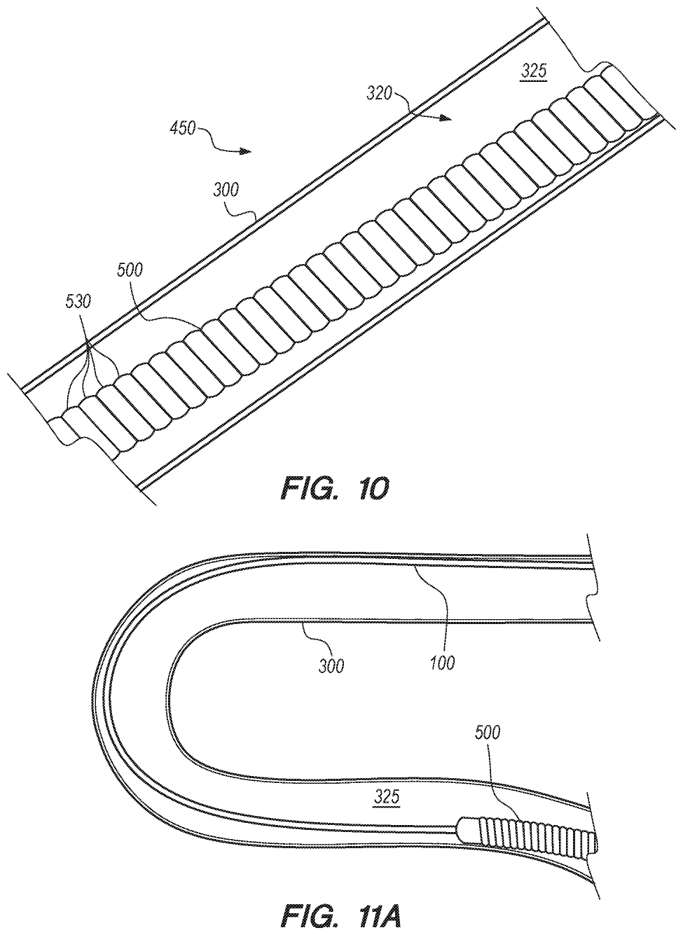

FIG. 10 illustrates a medical device 400 constructed in accordance with the features of FIGS. 8-9A. The medical device 400 includes a coil 500 made from an elongated member having a D-shape cross-section. The medical device 400 also includes a wire 100 (not shown), wherein the coil 500 is disposed over wire 100. The wire 100 with the coil 500 is disposed within a delivery catheter 300, which has an inner surface 320 defining a lumen 325. The coil 500 is tightly wound in close pitch having adjacent loops 530 contacting each other at their respective lateral surfaces 522 (schematically shown in FIG. 9). The close pitch of the coil 500 provides column strength, thereby allowing a desirable pushability to be achieved.

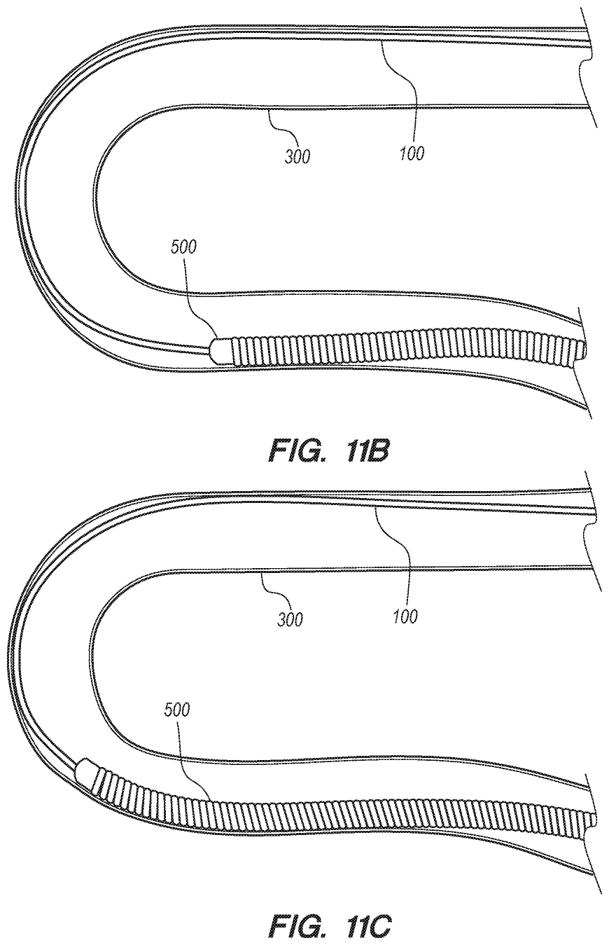

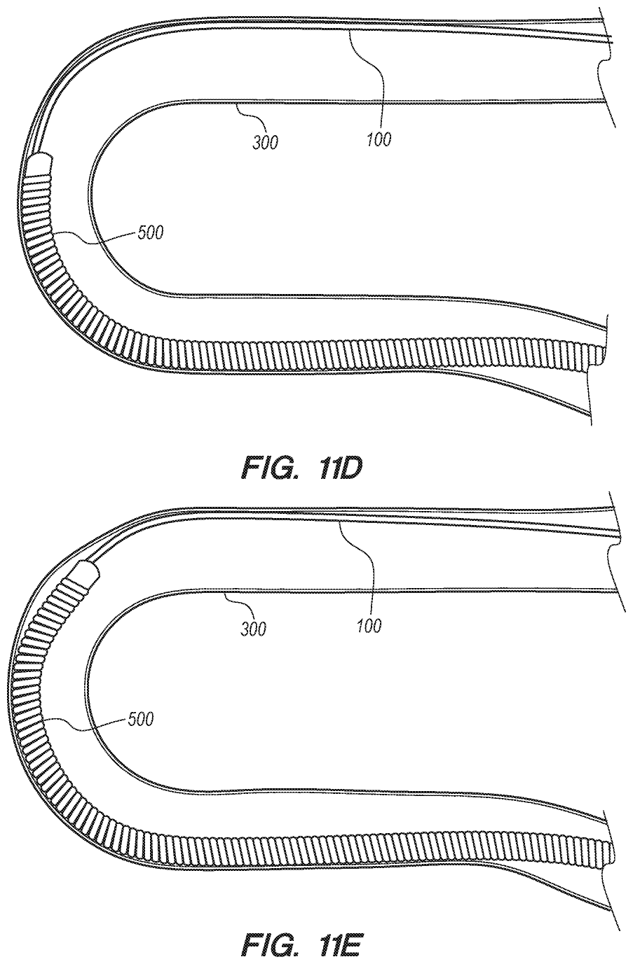

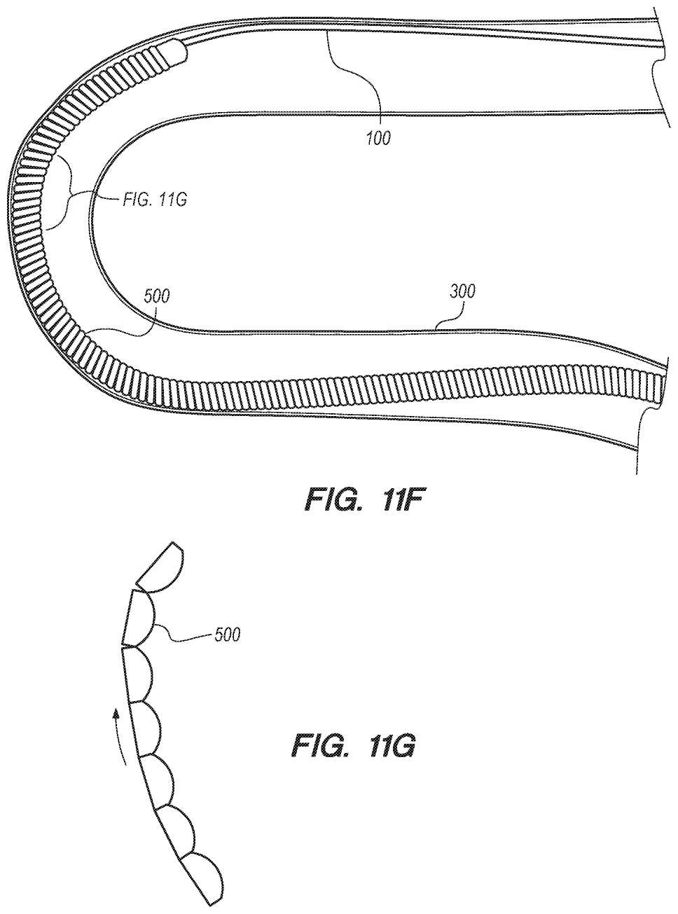

FIGS. 11A-11F illustrate a method of using the medical device 450 of FIG. 10. To use the medical device 450, an incision is first made at a patient's skin. Then the distal end of the medical device 450 is inserted through the incision to access a lumen of a blood vessel. In some embodiments, the medical device 450 may also include a catheter. In such cases, the catheter and the wire 100 with the coil 500 may be inserted into the blood vessel. In other embodiments, the wire 100 and the coil 500 may be inserted into the blood vessel without the catheter. Thus, in some embodiments, the wire 100 with the coil 500 may be advanced distally within a catheter, and in other embodiments, the wire 100 with the coil 500 may be advanced distally within a blood vessel without a catheter. The exemplary method will be described with reference to the wire 100 with the coil 500 being advanced in a catheter. However, it should be appreciated that the wire 100 with the coil 500 may be advanced inside a vessel without a catheter.

FIG. 11A shows the wire 100 with the coil 500 being advanced through the lumen 325 of the delivery catheter 300, where the coil 500 is at a relatively straight section of the delivery catheter 300. The wire 100 with the coil 500 may then be further advanced distally inside the blood vessel by applying pushing force at the proximal end 452 (shown schematically in FIG. 8) of the wire 100. FIG. 11B shows the coil 500 approaching a beginning of a tight bend. FIG. 11C shows the coil 500 being in the beginning of the tight bend. FIG. 11D shows the coil 500 reaching a mid-section of the tight bend. FIG. 11E shows the coil 500 reaching an end of the tight bend. FIG. 11F shows the coil 500 reaching another straight section of the delivery catheter 300.

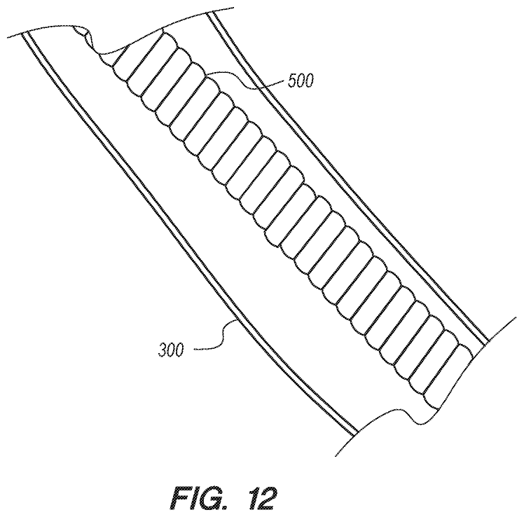

FIG. 12 illustrates the coil 500 of FIG. 10 after advancement within the delivery catheter 300. As shown in FIG. 12, the coil 500 having the D-shape cross-section 550 maintains a tight pitch winding and substantially maintains a uniform or smooth outer surface along the length of the coil 500.

As shown in FIG. 11G, the cross-section 550 of the elongated member 532 forming the coil 500 is advantageous because it prevents concentrated stress from being built up in the coil 500 as the coil undergoes bending, thereby avoiding formation of kink and plastic deformation. Thus, the coil 500 is able to form a smooth profile along a length of the wire 100 as the wire 100 undergoes bending so that no part of the coil body 506 forms any sharp bend or kink. The cross-section 550 also prevents shifting of the adjacent loops relative to each other, which may otherwise occur due to significant compression stress if the W:H ratio is less than 2:1 and or if the outer side of the cross-section 550 has right-angled corners. In particular, due to the thin profile of the cross-section 550 (i.e., W:H ratio being at least 2:1), the maximum compression stress due to bending of the coil 200 imposed on the coil 200 is reduced (compared to a cross-section with W:H ratio being less than 2:1). The lack of right-angled corners on the outside (facing away from the wire 100) of the cross-section 550 also assists in reducing the maximum compression stress caused by bending of the coil 200. Thus, adjacent ones of the loops 530 of the coil 500 are able to stay aligned as the wire 100 with the coil 500 undergoes bending. Furthermore, due to the low profile of the cross-section 550 (e.g., small height H compared to the width W), the wire 100 with the coil 500 remains sufficiently flexible so as to allow the wire 100 to navigate through tight bend of the catheter 300 and/or vessel. Furthermore, the cross-section 550 of the elongated member 532 allows stress associated with bending of the coil 500 to distribute to multiple loops 530 along the length of the coil 500, which further reduces risk of stress concentration along the coil 500 and reduces significant stretching of the coil 500 on the tension side of the cross-section 550 as the coil 500 is bending. The curvilinear side 540 of the cross-section 550 forming a smooth or non-sharp transition with the lateral side 520 is also advantageous because it allows the coil 500 to be smoothly advanced within the delivery catheter 300 or blood vessel, while avoiding or minimizing snagging, catching, abrading or the like, the inner surface of the catheter 300 or the vessel walls. In some cases, due to the smooth transition between the outward-facing side 540 and the lateral side 510 of the cross-section 550, even if loops 530 become misaligned during use, the misaligned loops 530 will not create serrated sharp corners or edges. Furthermore, the rectilinear profile of the lateral sides 520 of the cross-section 550 allows the coil 500 to bend in a more stable and predictable manner, because adjacent loops 530 can have more positional stability when they abut against each other along their respective lateral flat surfaces 522 (associated with the lateral sides 520). As a result there none of the loops 530 will move (e.g., roll) out of alignment to rest over its two adjacent loops 530 (which may otherwise occur for circular cross-section member). The coil 500 with the cross-section 550 is compatible with low-profile implant delivery systems, and provides a desirable flexibility, a desirable kink resistance, a desirable pushability, a desirable torqueability, or any combination of the foregoing.

In one or more embodiments described herein, the coil 500 may be configured to provide structural support for the wire 100 so that the wire 100 with the coil 500 (forming the medical device 450) has a desirable flexibility, a desirable kink-resistance, a desirable pushability, a desirable torqueability, or any combination of the foregoing. In some embodiments, a desirable flexibility and kink-resistance are considered to be achieved by the medical device 450 (e.g., a guidewire, a pushwire, a delivery wire, etc.) if the wire 100 and the coil 500 do not break, do not have any plastic deformation and kink, and/or do not form “serrated” sharp edges after having been delivered through a vasculature. In addition, in some embodiments, a desired torqueability is considered to be achieved by the medical device 450 if a twisting or torqueing motion applied at a proximal end about a longitudinal axis of the medical device 450 to turn the proximal end of the medical device 450 by an angle P will result in a turning of the distal end of the medical device 450 by an angle D that is at least 80% of P, or more preferably at least 90% of P, or even more preferably at least 95% of P (e.g., 100% of P, which means that the distal end of the medical device 450 has 1:1 response with respect to a torque applied at the proximal end of the medical device 450). Also, in some embodiments, a desired pushability may be achieved if the medical device 450 does not kink or buckle while being advanced inside a vessel or inside a catheter.

In addition, in one or more embodiments described herein, the wire 100 may be 50 to 300 cm in length, and together with the coil 500 may be 0.002 to 0.1 cm in diameter, depending upon the application. In other embodiments, the wire 100 may be shorter than 50 cm or longer than 300 cm in length. Also, in other embodiments, the wire 100 with the coil 500 may have a cross-sectional dimension that is larger than 0.1 cm, or less than 0.002 cm.

Also, in one or more embodiments described herein, the coil 500 may be made from any materials. By means of non-limiting examples, the coil may be made from stainless steel, alloys (e.g., Nitinol®, stainless steel, etc.), biocompatible metal, polymeric material(s) such as polyethylene, or any combinations thereof. In some embodiments, the coil 500 may be disposed around a portion of the wire 100. In other embodiments, the coil 500 may be disposed around the entire length of the wire 100. The pitch of the coil 500 may be varied along the length of the wire 100 to vary the stiffness of the wire 100. Also, in some embodiments, the medical device 450 may include multiple coils 500 disposed around the wire 100.

In one or more embodiments, the wire 100 and/or the coil 500 may include markings for purpose of imaging (e.g., markers composed of radio-opaque material(s)). In some embodiments, the wire 100 and/or the coil 500 may be made of radio-opaque material(s). In other embodiments, a radiopaque marker may be disposed on a distal portion of the coil 500. An epoxy bond may be used to attach the marker to the wire 100. The marker may be in a form of a band, but in other embodiments, the marker may have other shapes.

Also, in some embodiments, the coil 500 may be tightly wound around the wire 100. In other embodiments, the coil 500 may be loosely wound around the wire 100. In one or more embodiments, the coil 500 may have a tight pitch so that adjacent 530 loops of the coil 500 abut each other. In other embodiments, adjacent loops 530 of the coil 500 may be spaced away from each other.

The following items are exemplary features of embodiments described herein. Each item may be an embodiment itself or may be a part of an embodiment. One or more items described below may be combined with other item(s) in an embodiment.

Item 1: A medical device includes: a wire having a proximal end, a distal end, and a body extending between the proximal end and the distal end; and a coil disposed around a segment of the wire, wherein the coil comprises a proximal coil end, a distal coil end, and a coil body extending between the proximal coil end and the distal coil end; wherein the coil body comprises loops made from an elongated member having a cross-section, wherein the cross-section of the elongated member comprises a first side facing the wire, and a second side being opposite the first side and facing away from the wire; and wherein the second side of the cross-section of the elongated member has a higher curvature than the first side of the cross-section of the elongated member.

Item 2: At least a part of the first side comprises a rectilinear profile.

Item 3: At least a part of the second side comprises a curvilinear profile.

Item 4: The first side has a width, and the cross-section of the elongated member has a height that is perpendicular to the width, and wherein the width is at least two times the height.

Item 5: The height is a dimension of a lateral side of the cross-section, the lateral side having a rectilinear profile and is configured to abut against one of the loops of the coil.

Item 6: The cross-section of the elongated member comprises a D-shape cross-section.

Item 7: The coil body is configured to form a smooth profile along a length of the wire as the wire undergoes bending so that no part of the coil body forms any sharp bend, and/or adjacent ones of the loops of the coil are configured to stay aligned as the wire with the coil undergoes bending.

Item 8: The distal coil end of the coil is proximal to the distal end of the wire.

Item 9: The wire with the coil forms a guidewire.

Item 10: The wire with the coil forms a pushwire configured to push an object.

Item 11: The object comprises a stent or an embolic coil.

Item 12: A medical device includes: a wire having a proximal end, a distal end, and a body extending between the proximal end and the distal end; and a coil disposed around a segment of the wire, wherein the coil comprises a proximal coil end, a distal coil end, and a coil body extending between the proximal coil end and the distal coil end; wherein the coil body is configured to form a smooth profile along a length of the wire as the wire undergoes bending so that no part of the coil body forms any sharp bend, and/or wherein adjacent ones of the loops of the coil are configured to stay aligned as the wire with the coil undergoes bending.

Item 13: The coil body comprises loops made from an elongated member having a cross-section, wherein the cross-section of the elongated member comprises a first side facing the wire, and a second side being opposite the first side and facing away from the wire; and wherein the second side of the cross-section of the elongated member has a higher curvature than the first side of the cross-section of the elongated member.

Item 14: At least a part of the first side comprises a rectilinear profile.

Item 15: At least a part of the second side comprises a curvilinear profile.

Item 16: The first side has a width, and the cross-section of the elongated member has a height that is perpendicular to the width, and wherein the width is at least two times the height.

Item 17: The height is a dimension of a lateral side of the cross-section, the lateral side having a rectilinear profile and is configured to abut against one of the loops of the coil.

Item 18: The cross-section of the elongated member comprises a D-shape cross-section.

Item 19: The wire with the coil forms a pushwire configured to push an object, and wherein the object comprises a stent or an embolic coil.

Item 20: A medical device includes: a wire having a proximal end, a distal end, and a body extending between the proximal end and the distal end; and a coil disposed around a segment of the wire, wherein the coil comprises a proximal coil end, a distal coil end, and a coil body extending between the proximal coil end and the distal coil end; wherein the coil body comprises loops made from an elongated member having a cross-section, wherein the cross-section of the elongated member comprises a first side facing the wire, and a second side being opposite the first side and facing away from the wire; wherein the first side has a width, and the cross-section of the elongated member has a height that is perpendicular to the width, and wherein the width is at least two times the height; and wherein the height is a dimension of a lateral side of the cross-section, the lateral side having a rectilinear profile and is configured to abut against one of the loops of the coil.

Although particular embodiments have been shown and described herein, it will be understood by those skilled in the art that they are not intended to limit the disclosed inventions, and it will be obvious to those skilled in the art that various changes, permutations, and modifications may be made (e.g., the dimensions of various parts, combinations of parts) without departing from the scope of the disclosed inventions, which is to be defined only by the following claims and their equivalents. The specification and drawings are, accordingly, to be regarded in an illustrative rather than restrictive sense. The various embodiments shown and described herein are intended to cover alternatives, modifications, and equivalents of the disclosed inventions, which may be included within the scope of the appended claims.

What is claimed is:

1. A medical device, comprising:
a wire having a proximal end, a distal end, and a body extending between the proximal end and the distal end; and
a coil disposed around a segment of the wire, wherein the coil comprises a proximal coil end, a distal coil end, and a coil body extending between the proximal coil end and the distal coil end,
wherein the coil body comprises loops made from an elongated member having a cross-section, wherein the cross-section of the elongated member comprises a first side facing the wire, and a second side being opposite the first side and facing away from the wire, and
wherein the second side of the cross-section of the elongated member has a higher curvature than the first side of the cross-section of the elongated member.

2. The medical device of claim 1, wherein at least a part of the first side comprises a rectilinear profile.

3. The medical device of claim 1, wherein at least a part of the second side comprises a curvilinear profile.

4. The medical device of claim 1, wherein the first side has a width, and the cross-section of the elongated member has a height that is perpendicular to the width, and wherein the width is at greater than the height.

5. The medical device of claim 4, wherein the width is at least two times the height.

6. The medical device of claim 4, wherein the height is a dimension of a lateral side of the cross-section, the lateral side having a rectilinear profile and is configured to abut against one of the loops of the coil.

7. The medical device of claim 1, wherein the cross-section of the elongated member comprises a D-shape cross-section.

8. The medical device of claim 1, wherein the coil body is configured to form a smooth profile along a length of the wire as the wire undergoes bending so that no part of the coil body forms any sharp bend, and/or wherein adjacent ones of the loops of the coil are configured to stay aligned as the wire with the coil undergoes bending.

9. The medical device of claim 1, wherein the distal coil end of the coil is proximal to the distal end of the wire.

10. The medical device of claim 1, wherein the wire with the coil forms a guidewire.

11. The medical device of claim 1, wherein the wire with the coil forms a pushwire configured to push an object.

12. A medical device comprising:
a wire having a proximal end, a distal end, and a body extending between the proximal end and the distal end; and
a coil disposed around a segment of the wire, wherein the coil comprises a proximal coil end, a distal coil end, and a coil body extending between the proximal coil end and the distal coil end,
wherein the coil body is configured to form a smooth profile along a length of the wire as the wire undergoes bending so that no part of the coil body forms any sharp bend, and/or wherein adjacent ones of the loops of the coil are configured to stay aligned as the wire with the coil undergoes bending.

13. The medical device of claim 12, wherein the coil body comprises loops made from an elongated member having a cross-section comprising a first side facing the wire and a second side being opposite the first side and facing away from the wire, and wherein the second side of the cross-section of the elongated member has a higher curvature than the first side of the cross-section of the elongated member.

14. The medical device of claim 13, wherein at least a part of the first side comprises a rectilinear profile.

15. The medical device of claim 13, wherein at least a part of the second side comprises a curvilinear profile.

16. The medical device of claim 13, wherein the first side has a width, and the cross-section of the elongated member has a height that is perpendicular to the width, and wherein the width is at least two times the height.

17. The medical device of claim 16, wherein the height is a dimension of a lateral side of the cross-section, the lateral side having a rectilinear profile and is configured to abut against one of the loops of the coil.

18. The medical device of claim 13, wherein the cross-section of the elongated member comprises a D-shape cross-section.

19. The medical device of claim 12, wherein the wire with the coil forms a pushwire configured to push an object, and wherein the object comprises a stent or an embolic coil.

20. A medical device, comprising:
a wire having a proximal end, a distal end, and a body extending between the proximal end and the distal end; and
a coil disposed around a segment of the wire, wherein the coil comprises a proximal coil end, a distal coil end, and a coil body extending between the proximal coil end and the distal coil end,
wherein the coil body comprises loops made from an elongated member having a cross-section comprising a first side facing the wire and a second side being opposite the first side and facing away from the wire,
wherein the first side has a width, and the cross-section of the elongated member has a height that is perpendicular to the width, and wherein the width is at least two times the height, and
wherein the height is a dimension of a lateral side of the cross-section, the lateral side having a rectilinear profile and is configured to abut against one of the loops of the coil.
